SEQUENCE LISTING (36 sequences)

SEQ ID NO: 1 - RNA, 247 nt, Homo sapiens
  source 1..247: mol_type = mRNA
gtgatcctgctggaagaggcgtggagtgaactctttctcctcggggccatccagtggtctctgcctctggacagctgtcctctgctggcaccgcccgaggcctctgctgccggtggtgcccagggccggctcacgctggccagcatggagacgcgtgtcctgcaggaaactatctctcggttccgggcattggcggtggaccccacggagtttgcctgcatgaaggccttggtcctcttcaagccag

SEQ ID NO: 2 - RNA, 50 nt, Homo sapiens
  source 1..50: mol_type = mRNA
gcaggaaactatctctcggttccaggcattggcggtggaccccacggagt

SEQ ID NO: 3 - RNA, 20 nt, Synthetic construct
  source 1..20: mol_type = other RNA
accgagagatagtttcctgc

SEQ ID NO: 4 - RNA, 20 nt, Synthetic construct
  source 1..20: mol_type = other RNA
aaccgagagatagtttcctg

SEQ ID NO: 5 - RNA, 20 nt, Synthetic construct
  source 1..20: mol_type = other RNA
gaaccgagagatagtttcct

SEQ ID NO: 6 - RNA, 20 nt, Synthetic construct
  source 1..20: mol_type = other RNA
ggaaccgagagatagtttcc

SEQ ID NO: 7 - RNA, 20 nt, Synthetic construct
  source 1..20: mol_type = other RNA
tggaaccgagagatagtttc

SEQ ID NO: 8 - RNA, 20 nt, Synthetic construct
  source 1..20: mol_type = other RNA
ctggaaccgagagatagttt

SEQ ID NO: 9 - RNA, 20 nt, Synthetic construct
  source 1..20: mol_type = other RNA
cctggaaccgagagatagtt

SEQ ID NO: 10 - RNA, 20 nt, Synthetic construct
  source 1..20: mol_type = other RNA
gcctggaaccgagagatagt

SEQ ID NO: 11 - RNA, 20 nt, Synthetic construct
  source 1..20: mol_type = other RNA
tgcctggaaccgagagatag

SEQ ID NO: 12 - RNA, 20 nt, Synthetic construct
  source 1..20: mol_type = other RNA
atgcctggaaccgagagata

SEQ ID NO: 13 - RNA, 20 nt, Synthetic construct
  source 1..20: mol_type = other RNA
aatgcctggaaccgagagat

SEQ ID NO: 14 - RNA, 20 nt, Synthetic construct
  source 1..20: mol_type = other RNA
caatgcctggaaccgagaga

SEQ ID NO: 15 - RNA, 20 nt, Synthetic construct
  source 1..20: mol_type = other RNA
ccaatgcctggaaccgagag

SEQ ID NO: 16 - RNA, 20 nt, Synthetic construct
  source 1..20: mol_type = other RNA
gccaatgcctggaaccgaga

SEQ ID NO: 17 - RNA, 20 nt, Synthetic construct
  source 1..20: mol_type = other RNA
cgccaatgcctggaaccgag

SEQ ID NO: 18 - RNA, 20 nt, Synthetic construct
  source 1..20: mol_type = other RNA
ccgccaatgcctggaaccga

SEQ ID NO: 19 - RNA, 20 nt, Synthetic construct
  source 1..20: mol_type = other RNA
accgccaatgcctggaaccg

SEQ ID NO: 20 - RNA, 20 nt, Synthetic construct
  source 1..20: mol_type = other RNA
caccgccaatgcctggaacc

SEQ ID NO: 21 - RNA, 20 nt, Synthetic construct
  source 1..20: mol_type = other RNA
ccaccgccaatgcctggaac

SEQ ID NO: 22 - RNA, 20 nt, Synthetic construct
  source 1..20: mol_type = other RNA
tccaccgccaatgcctggaa

SEQ ID NO: 23 - RNA, 20 nt, Synthetic construct
  source 1..20: mol_type = other RNA
gtccaccgccaatgcctgga

SEQ ID NO: 24 - RNA, 20 nt, Synthetic construct
  source 1..20: mol_type = other RNA
ggtccaccgccaatgcctgg

SEQ ID NO: 25 - RNA, 20 nt, Synthetic construct
  source 1..20: mol_type = other RNA
gggtccaccgccaatgcctg

SEQ ID NO: 26 - RNA, 20 nt, Synthetic construct
  source 1..20: mol_type = other RNA
ggggtccaccgccaatgcct

SEQ ID NO: 27 - RNA, 20 nt, Synthetic construct
  source 1..20: mol_type = other RNA
tggggtccaccgccaatgcc

SEQ ID NO: 28 - RNA, 20 nt, Synthetic construct
  source 1..20: mol_type = other RNA
gtggggtccaccgccaatgc

SEQ ID NO: 29 - RNA, 20 nt, Synthetic construct
  source 1..20: mol_type = other RNA
cgtggggtccaccgccaatg

SEQ ID NO: 30 - RNA, 20 nt, Synthetic construct
  source 1..20: mol_type = other RNA
ccgtggggtccaccgccaat

SEQ ID NO: 31 - RNA, 20 nt, Synthetic construct
  source 1..20: mol_type = other RNA
tccgtggggtccaccgccaa

SEQ ID NO: 32 - RNA, 20 nt, Synthetic construct
  source 1..20: mol_type = other RNA
ctccgtggggtccaccgcca

SEQ ID NO: 33 - RNA, 20 nt, Synthetic construct
  source 1..20: mol_type = other RNA
actccgtggggtccaccgcc

SEQ ID NO: 34 - DNA, 20 bp, Synthetic construct
  source 1..20: mol_type = other DNA
cccctcctctccatactcct

SEQ ID NO: 35 - DNA, 20 bp, Synthetic construct
  source 1..20: mol_type = other DNA
cctctacgtgctcaggatcc

SEQ ID NO: 36 - RNA, 18 nt, Synthetic construct
  source 1..18: mol_type = other RNA
ttagtttaatcacgctcg

(19) United States
(12) Patent Application Publication
(10) Pub. No.: US 2025/0283093 A1
(43) Pub. Date: Sep. 11, 2025

(54) COMPOSITIONS AND METHODS FOR TREATING RETINAL DISEASES

(71) Applicant: SKIP THERAPEUTICS LTD., Ness Ziona (IL)

(72) Inventors: Ariel FEIGLIN, Mazkeret Batya (IL); Yehezkel SZTAINBERG, Rehovot (IL); Ilana BUCHUMENSKI, Ashkelon (IL); Maya DAVID TEITELBAUM, Rishon LeTsiyon (IL); Hagit T. PORATH, Moshav Gimzo (IL)

(21) Appl. No.: 19/130,536

(22) PCT Filed: Nov. 16, 2023
(86) PCT No.: PCT/IL2023/051187

§ 371 Date: May 15, 2025

Related U.S. Application Data
(60) Provisional application No. 63/426,077, filed on Nov. 17, 2022.

(51) Int. Cl. C12N 15/113

(52) U.S. Cl. CPC C12N 15/1138; C12N 2310/14; C12N 2320/33

(57) ABSTRACT

The present invention is directed to, inter alia, a method for treating retinal disease using a splicing modulator, such as an antisense oligonucleotide, capable of inducing the native splicing of exon 6 of a mutant nuclear receptor subfamily, 2 group, E member 3 (NR2E3) pre-mRNA. Also provided is a composition comprising the splicing modulator, and use of same.

### CROSS-REFERENCE TO RELATED APPLICATIONS

This application claims the benefit of priority of U.S. Provisional Patent Application No. 63/426,077, titled “COMPOSITIONS AND METHODS FOR TREATING RETINAL DISEASES”, filed 17 Nov. 2022, the contents of which are incorporated herein by reference in their entirety.

### REFERENCE TO AN ELECTRONIC SEQUENCE LISTING

The contents of the electronic sequence listing (SKIP-P-002-PCT ST26.xml; size: 32,896 bytes; and date of creation: Oct. 11, 2023) is herein incorporated by reference in its entirety.

### FIELD OF INVENTION

The present invention is in the field of antisense oligonucleotides and therapeutic use of the antisense oligonucleotides.

### BACKGROUND

Functional restoration of gene function in genetic disorders has recently been achieved by modulating splicing of the mutated genes using antisense oligonucleotides (ASOs). ASOs are single stranded, chemically modified, nucleic acids that bind pre-mRNA of the target gene and alter splicing in a way that can restore gene functionality. For example, Nusinersen, is an ASO-based drug that changes the splicing pattern of SMN2 and is successfully used for treatment of spinal muscular atrophy (SMA, Parkash 2107; Gene Ther). Another example is Milasen, an “N=1” drug, where an ASO was developed to effectively restore correct splicing for a unique mutation in the MFSD8 gene that lead to batten disease (Kim et al., 2019; N Engl J Med).

Deficiency in the nuclear receptor subfamily, 2 group, E member 3 (NR2E3) gene causes a range of inherited retinal disorders including enhanced S-cone syndrome (ESCS), retinitis pigmentosa, Goldmann-Favre syndrome (GFS), and clumped pigmentary retinal degeneration (CPRD) (Mollema and Haider 2010; Exp Eye Res). NR2E3 is a member of a superfamily of nuclear receptors that regulate transcription in a ligand-dependent manner. NR2E3 expression is restricted to retinal photoreceptors in human and is likely to function in multiple capacities: 1) as an activator directing post-mitotic cells to differentiate into rods; 2) as a repressor affecting retinal progenitor competency and retinal development; and 3) a regulator of phototransduction in adult rods and cones (Mollema and Haider 2010; Exp Eye Res).

A relatively frequent disease-causing mutation in NR2E3 is c.932G>A (p.Arg311Gln) (Bandah et al., 2009; Arch Ophthalmol, ClinVarID: 5532). This mutation is classified as a missense variant, meaning that it causes a change of the arginine residue at position 311 to a glutamine residue. The mechanism by which this change causes the apparent disease phenotype is not fully understood (see Roduit et al., 2009; PLOS One, where it is demonstrated that this residue change does not affect homodimerization, interaction with CRX co-repressor and DNA binding).

There is still a great need for compounds suitable for treating or ameliorating a retinal disease specifically in a subject harboring the c.932G>A mutation.

### SUMMARY

The present invention, in some embodiments, is directed to a method of splicing modulation of the NR2E3 gene such that effect of a disease-causing mutation is eliminated or lessened.

The present invention, in some embodiments, is based, at least in part, on the finding that the c.932G>A mutation triggers aberrant splicing of the NR2E3 transcript, thus, leading to an in-frame loss of 186 bases in exon 6 (corresponding to 62 amino acids in the protein product encoded from the NR2E3 transcript). Consequently, the inventors suggest using splice shifting/modifying antisense oligonucleotides (ASOs) so as to restore the native and/or proper splicing of the NR2E3 transcript, and thereby, restore functionality of the protein product encoded therefrom.

According to one aspect, there is provided a method for treating a retinal disease in a subject in need thereof, the method comprising administering to the subject a therapeutically effective amount of at least one synthetic antisense oligonucleotide (ASO), wherein the ASO induces the inclusion of nucleotides in positions 748-933 of the nuclear receptor subfamily, 2 group, E member 3 (NR2E3) pre-mRNA, thereby treating a retinal disease in the subject.

According to another aspect, there is provided a composition comprising an ASO comprising 14 to 25 bases having at least 80% complementarity to a NR2E3 pre-mRNA and characterized by inducing inclusion of nucleotides in positions 748-933 of the NR2E3 pre-mRNA.

A method for producing a compound suitable for treating a retinal disease, the method comprising a compound that binds to exon 6 of the NR2E3 pre-mRNA, assaying the inclusion of nucleotides in positions 748-933 of the NR2E3 pre-mRNA in the presence of the compound, and selecting at least one compound that induces the inclusion of nucleotides in positions 748-933 of the NR2E3 pre-mRNA.

In some embodiments, the ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the ASO comprises 14 to 25 bases.

In some embodiments, the ASO has at least 75% complementarity to an equal-length portion of a nucleic acid sequence derived from the polynucleotide sequence:

(SEQ ID NO: 1)
GTGATCCTGCTGGAAGAGGCGTGGAGTGAACTCTTTCTCCTCGGGGCC
ATCCAGTGGTCTCTGCCTCTGGACAGCTGTCCTCTGCTGGCACCGCCC
GAGGCCTCTGCTGCCGGTGGTGCCCAGGGCCGGCTCACGCTGGCCAGC
ATGGAGACGCGTGTCCTGCAGGAAACTATCTCTCGGTTCCGGGCATTG
GCGGTGGACCCCACGGAGTTTGCCTGCATGAAGGCCTTGGTCCTCTTC
AAGCCAG.

In some embodiments, the ASO has at least 75% complementarity to

(SEQ ID NO: 2)
GCAGGAAACTATCTCTCGGTTCCAGGCATTGGCGGTGGACCCCACGGA
GT.

In some embodiments, the subject comprises at least one in-frame and/or missense mutation in exon 6 of NR2E3.

In some embodiments, the at least one mutation is c.932G>A.

In some embodiments, the ASO comprises a chemically modified backbone.

In some embodiments, the chemically modified backbone comprises: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

In some embodiments, the composition further comprises a pharmaceutically acceptable carrier.

In some embodiments, the is for use in inducing the inclusion of nucleotides in positions 748-933 of the NR2E3 pre-mRNA, in a subject in need thereof.

In some embodiments, the subject comprises a c.932G>A mutation in exon 6 of the NR2E3 pre-mRNA.

In some embodiments, the subject is afflicted with or at increased risk for developing a retinal disease.

In some embodiments, the composition is for use in treatment or prevention of a retinal disease in a subject in need thereof.

In some embodiments, the NR2E3 pre-mRNA comprises a c.932G>A mutation in exon 6.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1E include micrographs, fluorescent micrographs, non-limiting diagrams, and a chromatogram showing that aberrant splicing of the NR2E3 pre-mRNA is caused by the p.Arg311Gln mutation, in HEK293 cells. (1A) Gel electrophoresis of RT-PCR products obtained from HEK293 cells transfected with NR2E3^{WT} or NR2E3^{p.Arg311Gln} plasmids (flag-tagged). RT-PCR was performed using primers spanning exons 5, 6, and 7. (1B) A diagram showing the aberrant splicing caused by the p.Arg311Gln mutation in exon 6. Sequencing of the lower band of the RT-PCR product of NR2E3^{p.Arg311Gln} confirm the predicted aberrant splicing. (1C) Protein was extracted from cells 48 hr post-transfection, and lysates were analyzed by western blot using anti-flag antibody. Histone 3 (H3) is used as a loading control. (1D) Immunofluorescence staining with anti-flag antibody reveals loss of nuclear localization of NR2E3^{9.Arg311Gln}, compared to NR2E3^{WT}. (1E) Non-limiting potential therapeutic correction strategy-using ASOs to block aberrant splicing in attempt to restore native and proper splicing.

FIGS. 2A-2C include an image, photographs and a vertical bar graph showing identification of ASOs that block aberrant splicing caused by the c.932G>A mutation in NR2E3 and restore normal splicing. (2A) Snapshot image from the UCSC browser showing exon 6 of the NR2E3 gene and the genomic position of ASOs 1-16 (SEQ ID NOs: 3, 5, 7, 9, 11, 13, 15, 17, 19, 21, 23, 25, 27, 29, 31 and 33). NR2E3 c.932G>A mutation position marked with a solid vertical line. Gray shading indicates the remaining portion of exon 6 due to aberrant splicing caused by the mutation (2B) ASO mini-screen targeting NR2E3 exon 6. 16 ASOs were evaluated in HEK293 cells transfected with NR2E3^{c.932G>A} plasmid. RT-PCR results are shown using primers for exons 5, and 7 (bottom panel) (Forward sequence: 5′-CCCCTCCTCTCCATACTCCT-3′ (SEQ ID NO: 34), Reverse sequence: 5′-CCTCTACGTGCTCAGGATCC-3′; (SEQ ID NO: 35)). Ctrl-antisense control (TTAGTTTAATCACGCTCG; (SEQ ID NO: 36)). pWT-plasmid expressing NR2E3WT. (2C) Western blot and densitometry analysis confirm ASO-2 (SEQ ID NO: 4) correction of aberrant splicing at the protein level. GAPDH is used as a loading control.

FIGS. 3A-3C include fluorescent micrographs, a photograph and vertical bar graphs showing that ASO treatment corrects mutated NR2E3^{c.932G>A} protein sub-cellular localization and Rhodopsin (RHO) promoter activation. (3A) Immunofluorescence staining following transfection of plasmids and ASO treatment in HeLa cells. (3B) Western blot analysis (left panel) following nucleus-cytoplasm fractionation of HEK293 cells transfected with plasmids and treated with ASO. H3 and GAPDH are used to confirm correct nucleus-cytoplasm fractionation. Right panel, densitometric quantification of restored nuclear NR2E3, highlighted in the blot with a red dashed rectangle. (3C) Luciferase activity assay following ASO treatment (n=4). All groups were transfected with RHO reporter, CRX, and NRL. ns—not significant. Ctrl-ASO control (as above). Data are mean±s.e.m. *P<0.05; **P<0.01 (One-way ANOVA).

### DETAILED DESCRIPTION

### Method of Treatment

According to some embodiments, a method for treating a retinal diseases in a subject is provided. In some embodiments, the method comprises administering to the subject a therapeutically effective amount of a splicing modulator, wherein the splicing modulator induces the endogenous splicing of exon 6 of the nuclear receptor subfamily, 2 group, E member 3 (NR2E3) pre-mRNA, thereby treating a retinal disease in the subject.

According to some embodiments, there is provided a method for inducing full or complete inclusion of exon 6 in the mature mRNA product of NR2E3.

According to some embodiments, there is provided a method for inducing inclusion of nucleotides in positions 748-933 of exon 6 of the NR2E3 pre-RNA in the mature mRNA product of NR2E3.

As used herein, the term “full or complete inclusion of exon 6” refers to a spliced or mature mRNA of the NR2E3 product comprising nucleotides 1-186 of exon 6.

In some embodiments, a full or complete inclusion of exon 6 results in a spliced or mature mRNA of the NR2E3 product comprising nucleotides 1-1,240 of the NR2E3 mRNA.

In some embodiments, a full or complete inclusion of exon 6 results in a spliced or mature mRNA of the NR2E3 product comprising at least 1,200, at least 1210, at least 1,220 or at least 1,230 nucleotides, or any value and range therebetween. Each possibility represents a separate embodiment of the invention.

In some embodiments, a protein product encoded from a spliced or mature mRNA of the NR2E3 gene that fully or completely includes exon 6, as obtained by the instant application, comprises 410 amino acids. In some embodiments, the protein product comprises amino acids encoded from positions 1-186 of exon 6 of the NR2E3 mRNA. In some embodiments, the protein product comprises amino acids in positions 250-311. In some embodiments, a mutated NR2E3 gene, as disclosed herein, is transcribed into an mRNA wherein exon 6 is partially excluded, thus giving rise to an NR2E3 protein being devoid of amino acids in positions 250-311 of the wildtype NR2E3 protein.

In some embodiments, the protein product comprises arginine to glutamine substitution in position 311 of the protein.

In some embodiments, the spliced or mature mRNA of the NR2E3 gene, obtained according to the instant application comprises at least 1,200, at least 1210, at least 1,220 or at least 1,230 nucleotides, or any value and range therebetween, and a G932A substitution. Each possibility represents a separate embodiment of the invention.

According to some embodiments, there is provided a method for suppressing or inhibiting exclusion of a portion of exon 6 of the NR2E3 pre-RNA from the mature mRNA product of NR2E3.

According to some embodiments, there is provided a method for suppressing or inhibiting exclusion of nucleotides in positions 748-933 of NR2E3 pre-RNA from the mature mRNA product of NR2E3.

In some embodiments, endogenous splicing of exon 6 comprises inclusion of exon 6 in the mature mRNA product of NR2E3.

In some embodiments, the splicing modulator induces full or complete inclusion of exon 6 in the mature mRNA product of NR2E3.

In some embodiments, the splicing modulator induces inclusion of nucleotides in positions 748-933 of the mature mRNA product of NR2E3.

In some embodiments, the splicing modulator is capable of binding to a pre-mRNA of the NR2E3 gene, and inducing or promoting full or complete inclusion of exon 6 in the mature NR2E3 mRNA.

In some embodiments, the splicing modulator is capable of binding to a pre-mRNA of the NR2E3 gene, and inducing or promoting inclusion of nucleotides in positions 748-933 of the mature NR2E3 mRNA.

In some embodiments, the splicing modulator is capable of binding to a pre-mRNA of the NR2E3 gene, and suppressing exclusion of nucleotides in positions 748-933 of the mature NR2E3 mRNA.

In some embodiments, nucleotides in positions 748-933 of the mature NR2E3 mRNA, are located in exon 6 of the NR2E3 pre-mRNA.

In some embodiments, a retinal disease is or comprises an inherited retina disease.

In some embodiments, a retinal disease is a genetic retinal disease.

In some embodiments, a genetic retinal disease is induced by or involves a mutation inducing at least a partial exclusion of exon 6 of the NR2E3 mRNA.

In some embodiments, a genetic retinal disease is induced by or involves a mutation inducing at least a partial exclusion of nucleotides in positions 748-933 of the NR2E3 mRNA.

In some embodiments, a genetic retinal disease is induced by or involves a mutation resulting in a short NR2E3 protein compared to the wildtype NR2E3 protein.

In some embodiments, the short NR2E3 protein comprises 348 amino acids. In some embodiments, the short NR2E3 protein is devoid of or missing 62 amino acids encoded from nucleotides in positions 748-933 of the NR2E3 mRNA.

In some embodiments, the wildtype NR2E3 protein comprises 410 amino acids.

In some embodiments, the method comprises administering a splicing modulator which is at least one synthetic antisense oligonucleotide (ASO).

In some embodiments, the ASO is chemically modified. In some embodiments, the chemical modification is a modification of a backbone of the ASO. In some embodiments, the chemical modification is a modification of a sugar of the ASO. In some embodiments, the chemical modification is a modification of a nucleobase of the ASO. In some embodiments, the chemical modification increases stability of the ASO in a cell. In some embodiments, the chemical modification increases stability of the ASO in vivo. In some embodiments, the chemical modification increases the ASO's ability to modulate splicing. In some embodiments, the chemical modification increases the ASO's ability to induce full or complete inclusion of exon 6 of the NR2E3 pre-mRNA. In some embodiments, the chemical modification increases the ASO's ability to induce inclusion of nucleotides in positions 748-933 of the NR2E3 pre-mRNA. In some embodiments, the chemical modification increases the ASO's ability to suppress exclusion of nucleotides in positions 748-933 from the mature NR2E3 mRNA. In some embodiments, the chemical modification increases the half-life of the ASO. In some embodiments, the chemical modification inhibits polymerase extension from the 3′ end of the ASO. In some embodiments, the chemical modification inhibits recognition of the ASO by a polymerase. In some embodiments, the chemical modification inhibits double-strand trigged degradation. In some embodiments, the chemically modified ASO does not trigger nucleic acid double-stranded degradation upon binding a NR2E3 pre-mRNA. In some embodiments, the chemical modification inhibits RISC-mediated degradation. In some embodiments, the chemical modification inhibits RISC-mediated degradation or any parallel nucleic acid degradation pathway.

In some embodiments, the ASO is devoid of a labeling moiety. In some embodiments, the ASO is not labeled. In some embodiments, the ASO does not emit a detectable signal or does not comprise moieties capable of being recognized so as to enable nucleic acid detection (e.g., digoxigenin and fluorescently labeled anti-DIG antibody). In some embodiments, a detectable signal comprises a dye or an emitting energy which provides detection of a compound, e.g., a polynucleotide, in vivo or in vitro. In some embodiments, a detectable signal comprises: a fluorescent signal, a chromatic signal, or a radioactive signal.

In some embodiments, the ASO is devoid of radioactive nucleobase(s); digoxigenin, streptavidin, biotin, a fluorophore, hapten label, CLICK label, amine label, or thiol label.

In some embodiments, the chemical modification is selected from: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, or any combination thereof.

In some embodiments, the ASO comprises at least 14 bases, at least 15 bases, at least 16 bases, at least 17 bases, at least 18 bases, at least 19 bases, at least 20 bases, at least 21 bases, at least 22 bases, at least 23 bases, at least 24 bases, or at least 25 bases, or any value and range therebetween. Each possibility represents a separate embodiment of the invention.

In some embodiments, the ASO comprises 14 to 30 bases, 14 to 28 bases, 14 to 26 bases, 14 to 24 bases, 14 to 21 bases, 14 to 19 bases, 14 to 18 bases, or 14 to 17 bases. Each possibility represents a separate embodiment of the invention. in some embodiments, the ASO comprises 17 to 22 bases.

In some embodiments, the ASO is complementary to exon 6 of the NR2E3 pre-mRNA.

In some embodiments, exon 6 of the NR2E3 pre-mRNA comprises the sequence:

(SEQ ID NO: 1)
GTGATCCTGCTGGAAGAGGCGTGGAGTGAACTCTTTCTCCTCGGGGCC
ATCCAGTGGTCTCTGCCTCTGGACAGCTGTCCTCTGCTGGCACCGCCC
GAGGCCTCTGCTGCCGGTGGTGCCCAGGGCCGGCTCACGCTGGCCAGC
ATGGAGACGCGTGTCCTGCAGGAAACTATCTCTCGGTTCCGGGCATTG
GCGGTGGACCCCACGGAGTTTGCCTGCATGAAGGCCTTGGTCCTCTTC
AAGCCAG.

In some embodiments, the ASO is complementary to an equal-length portion of a nucleic acid sequence derived from the polynucleotide sequence comprising or consisting of: GCAGGAAACTATCTCTCGGTTCCAGGCATTGGCGGTGGACCCCACGGAGT (SEQ ID NO: 2).

In some embodiments, the ASO has at least 75%, at least 80%, at least 85%, at least 90%, at least 95%, at least 99%, or 100% complementarity to an equal-length portion of a nucleic acid sequence derived from SEQ ID Nos: 1-2, any value and range therebetween. Each possibility represents a separate embodiment of the invention. In some embodiments, the ASO has 70-80%, 75-85%, 80-90%, 85-95%, 90-99%, or 95-100% complementarity to an equal-length portion of a nucleic acid sequence derived from SEQ ID Nos: 1-2. Each possibility represents a separate embodiment of the invention.

The term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated.

In some embodiments, the ASO comprises the nucleic acid sequence of:

(SEQ ID NO: 3)
ACCGAGAGATAGTTTCCTGC;
(SEQ ID NO: 4)
AACCGAGAGATAGTTTCCTG;
(SEQ ID NO: 5)
GAACCGAGAGATAGTTTCCT;
(SEQ ID NO: 6)
GGAACCGAGAGATAGTTTCC;
(SEQ ID NO: 7)
TGGAACCGAGAGATAGTTTC;
(SEQ ID NO: 8)
CTGGAACCGAGAGATAGTTT;
(SEQ ID NO: 9)
CCTGGAACCGAGAGATAGTT;
(SEQ ID NO: 10)
GCCTGGAACCGAGAGATAGT;
(SEQ ID NO: 11)
TGCCTGGAACCGAGAGATAG;
(SEQ ID NO: 12)
ATGCCTGGAACCGAGAGATA;
(SEQ ID NO: 13)
AATGCCTGGAACCGAGAGAT;
(SEQ ID NO: 14)
CAATGCCTGGAACCGAGAGA;
(SEQ ID NO: 15)
CCAATGCCTGGAACCGAGAG;
(SEQ ID NO: 16)
GCCAATGCCTGGAACCGAGA;
(SEQ ID NO: 17)
CGCCAATGCCTGGAACCGAG;
(SEQ ID NO: 18)
CCGCCAATGCCTGGAACCGA;
(SEQ ID NO: 19)
ACCGCCAATGCCTGGAACCG;
(SEQ ID NO: 20)
CACCGCCAATGCCTGGAACC;
(SEQ ID NO: 21)
CCACCGCCAATGCCTGGAAC;
(SEQ ID NO: 22)
TCCACCGCCAATGCCTGGAA;
(SEQ ID NO: 23)
GTCCACCGCCAATGCCTGGA;
(SEQ ID NO: 24)
GGTCCACCGCCAATGCCTGG;
(SEQ ID NO: 25)
GGGTCCACCGCCAATGCCTG;
(SEQ ID NO: 26)
GGGGTCCACCGCCAATGCCT;
(SEQ ID NO: 27)
TGGGGTCCACCGCCAATGCC;
(SEQ ID NO: 28)
GTGGGGTCCACCGCCAATGC;
(SEQ ID NO: 29)
CGTGGGGTCCACCGCCAATG;
(SEQ ID NO: 30)
CCGTGGGGTCCACCGCCAAT;
(SEQ ID NO: 31)
TCCGTGGGGTCCACCGCCAA;
(SEQ ID NO: 32)
CTCCGTGGGGTCCACCGCCA;
or
(SEQ ID NO: 33)
ACTCCGTGGGGTCCACCGCC.

In some embodiments, the pre-mRNA is a wildtype pre-mRNA. In some embodiments, the pre-mRNA is a mutated pre-mRNA. In some embodiments, the NR2E3 pre-mRNA comprises any one of: SEQ ID Nos: 1-2. In some embodiments, the ASO is complementary to a nucleic acid sequence comprising any one of: SEQ ID Nos: 1-2.

In some embodiments, the ASO comprises an active fragment of any one of SEQ ID Nos: 3-33.

As used herein, the term “active fragment” refers to a fragment that is 100% identical to a contiguous portion of the full nucleotide sequence of the ASO, providing that at least: 30%, 40%, 50%, 60%, 70%, 80% or 90% of the activity of the original ASO nucleotide sequence is retained, or any value and range therebetween. Each possibility represents a separate embodiment of the present invention.

In some embodiments, the ASO is specific to a NR2E3 pre-mRNA.

As used herein, the term “specific” refers to both base pair specificity and also gene specificity. In some embodiments, the ASO is specific to the NR2E3 gene. In some embodiments, the ASO is specific to a splice activating motif in NR2E3. In some embodiments, the ASO is specific to a splice activating region of NR2E3. In some embodiments, the splice activating is splice activating of a part or a portion of exon 6 of NR2E3.

In some embodiments, the ASO binds the NR2E3 pre-mRNA with perfect complementarity. In some embodiments, the ASO does not bind any gene or pre-mRNA product thereof, other than NR2E3 with perfect complementarity. In some embodiments, the ASO does not bind any gene or pre-mRNA product thereof, other than NR2E3 with a complementarity of greater than 70, 75, 80, 85, 90, 95, 97, 99 or 100%. Each possibility represents a separate embodiment of the invention. In some embodiments, the ASO does not bind any gene or pre-mRNA product thereof, other than NR2E3 with a complementarity of greater than 90%. In some embodiments, the ASO binds any one of: SEQ ID Nos: 1-2 with perfect complementarity. In some embodiments, the ASO does not bind any sequence other than SEQ ID Nos: 1-2 with complementarity of greater than 70, 75, 80, 85, 90, 95, 97, 99 or 100%. Each possibility represents a separate embodiment of the invention. In some embodiments, the ASO does not bind any sequence other than SEQ ID Nos: 1-2 with a complementarity of greater than 90%. In some embodiments, the ASO does not bind with perfect complementarity to anywhere in the genome or transcriptome (including pre-transcriptome, e.g., transcriptome comprising or consisting of pre-mRNA) of a cell other than within NR2E3. In some embodiments, the ASO does not bind with complementarity of greater than 70, 75, 80, 85, 90, 95, 97, 99 or 100% to anywhere in the genome or transcriptome (including pre-transcriptome, e.g., transcriptome comprising or consisting of pre-mRNA) of a cell other than within NR2E3. Each possibility represents a separate embodiment of the invention. In some embodiments, the cell is a mammalian cell. In some embodiments, the mammal is a human.

In some embodiments, the ASO modulates expression of NR2E3. In some embodiments, the ASO modulates splicing of NR2E3. In some embodiments, the ASO modulates splicing of exon 6 of NR2E3. In some embodiments, the ASO does not cause an off-target effect. In some embodiments, off-target is a target other than NR2E3. In some embodiments, off-target is a target other than splicing of exon 6 of NR2E3. In some embodiments, the ASO does not substantially or significantly modulate expression of a gene other than NR2E3. In some embodiments, the ASO does not substantially or significantly modulate splicing of a gene other than NR2E3. In some embodiments, the ASO does not substantially or significantly modulate splicing of an exon other than exon 6 of NR2E3. In some embodiments, substantial modulation of expression is a change in expression of at least 5, 10, 15, 20, 25, 30, 35, 40, 45 or 50%. Each possibility represents a separate embodiment of the invention. In some embodiments, substantial modulation of expression is a change in expression of at least 20%.

In some embodiments, the ASO is complementary to an exon-intron junction. In some embodiments the exon is exon 6 of the NR2E3 pre-mRNA. In some embodiments, the ASO is complementary to an aberrant splice junction residing within exon 6 of the NR2E3 pre-mRNA. In some embodiments, the aberrant splice junction within exon 6 of the NR2E3 comprises a substitution or mutation of G932A of the NR2E3 gene.

In some embodiments, the ASO is at least 70%, at least 80%, at least 85%, at least 90%, at least 95%, at least 99%, or 100% complementary to an aberrant splice junction as disclosed herein, or any value and range therebetween. Each possibility represents a separate embodiment of the invention. In some embodiments, the ASO is 70-85%, 80-90%, 85-95%, 90-99%, or 95-100% complementary to an aberrant splice junction as disclosed herein. Each possibility represents a separate embodiment of the invention.

In some embodiments, an ASO as disclosed herein targets, complements, induces, or any combination thereof, the full or complete inclusion of exon 6 of NR2E3 pre-mRNA transcribed from a mutated allele of the NR2E3 gene.

In some embodiments, an ASO as disclosed herein targets, complements, induces, or any combination thereof, the inclusion of nucleotides in positions 748 to 933 of exon 6 of NR2E3 pre-mRNA transcribed from a mutated allele of the NR2E3 gene.

In some embodiments, the subject comprises or is characterized by having a genome comprising at least one mutation in exon 6 of NR2E3 rendering a partially or fully non-functional NR2E3 protein.

In some embodiments, the subject comprises or is characterized by having a genome comprising at least one mutation in exon 6 of NR2E3 resulting in splicing and exclusion of nucleotides in positions 748 to 933 of exon 6 of NR2E3 pre-mRNA transcribed therefrom.

In some embodiments, the substitution or mutation as disclosed herein is an in-frame mutation. In some embodiments, the substitution or mutation as disclosed herein is a missense mutation.

In some embodiments, the at least one mutation is c.932G>A (also referred to herein as “G932A substitution”).

In some embodiments, a mutation as disclosed herein provides an arginine to glutamine substitution.

In some embodiments, “a mutation” as used herein, refers to a nucleotide substitution or modification which induces or results in a “retinal disease” in a subject harboring or comprising the mutation.

As used herein, the term “retinal disease” encompasses any symptom or manifestation related to diseases involving the retina. Methods for diagnosing retinal disease and/or symptoms associated therewith are common and would be apparent to one of ordinary skill in the art.

In some embodiments, the method is directed to improving at least one clinical parameter of a retinal disease in the subject.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “condition” includes anatomic and physiological deviations from the normal that constitute an impairment of the normal state of the living animal or one of its parts, that interrupts or modifies the performance of the bodily functions.

As used herein, the terms “subject” or “individual” or “animal” or “patient” or “mammal,” refers to any subject, particularly a mammalian subject, for whom therapy is desired, for example, a human.

In some embodiments, there is provided a method for treating retina disease in a subject in need thereof, comprising administering to the subject a therapeutically effective amount of a synthetic antisense oligonucleotide (ASO), wherein the ASO induces the full or complete inclusion of exon 6 of the NR2E3 pre-mRNA, thereby treating the retinal disease in the subject.

In some embodiments, the ASO induces inclusion of nucleotides in positions 748-933 of the NR2E3 pre-mRNA, thereby treating the retinal disease in the subject.

In some embodiments, there is provided a method for treating retina disease in a subject in need thereof, comprising administering to the subject a therapeutically effective amount of a synthetic antisense oligonucleotide (ASO), wherein the ASO suppresses the exclusion of a portion of exon 6 from the NR2E3 pre-mRNA, thereby treating the retinal disease in the subject.

In some embodiments, the ASO suppresses the exclusion of nucleotides in positions 748-933 of the NR2E3 pre-mRNA, thereby treating the retinal disease in the subject.

### Composition

According to some embodiments, there is provided a composition comprising an ASO comprising 14 to 30 bases having at least 80% complementarity to a NR2E3 pre-mRNA and characterized by inducing inclusion of nucleotides in positions 748-933 of the NR2E3 pre-mRNA.

According to some embodiments, there is provided a composition comprising an ASO comprising 14 to 30 bases having at least 80% complementarity to a NR2E3 pre-mRNA and characterized by suppressing exclusion of nucleotides in positions 748-933 of the NR2E3 pre-mRNA.

In some embodiments, the composition further comprises a pharmaceutically acceptable carrier.

The term “pharmaceutically acceptable carrier” as used herein refers to any of the standard pharmaceutical carriers known in the field such as sterile solutions, tablets, coated tablets, and capsules. Typically, such carriers contain excipients such as starch, milk, sugar, certain types of clay, gelatin, stearic acids, or salts thereof, magnesium or calcium stearate, talc, vegetable fats or oils, gums, glycols, or other known excipients. Such carriers may also include flavor and color additives or other ingredients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, nano-particles, nano-emulsions, pastes, creams, salves, etc., and may be in unit-dosage forms suitable for administration of relatively precise dosages.

In some embodiments, the pharmaceutical composition is formulated for systemic administration. In some embodiments, the pharmaceutical composition is formulated for administration to a subject. In some embodiments, the subject is a human subject. It will be understood by a skilled artisan that a pharmaceutical composition intended to administration to a subject should not have off-target effects, e.g., effects other than the intended therapeutic ones. In some embodiments, the pharmaceutical composition is devoid of a substantial effect on a gene other than NR2E3. In some embodiments, the pharmaceutical composition is devoid of a substantial effect on splicing of an exon other than exon 3 of NR2E3. In some embodiments, a substantial effect is one with a phenotypic result. In some embodiments, a substantial effect is a deleterious effect. In some embodiments, deleterious is with respect to the health and/or wellbeing of the subject.

In some embodiments, the composition is administered via a route selected from: topical administration, local administration, ocular administration, retinal administration, ophthalmic administration, systemic administration, intravitreal administration, or any combination thereof. In some embodiments, the composition is formulated for: topical administration, local administration, ocular administration, retinal administration, ophthalmic administration, systemic administration, intravitreal administration, or any combination thereof inhalation composition. In some embodiments, an ASO as disclosed and as described hereinabove, or a pharmaceutical composition comprising thereof, is used in the modulation of splicing of a NR2E3 pre-mRNA transcribed from a NR2E3 gene having a mutated exon 6.

The phrase “modulation of splicing” as used herein refers to affecting a change in the level of any RNA or mRNA variant produced by the NR2E3 native, mutated, or both, pre-mRNA.

In certain embodiments, the use is for increasing the level of an mRNA molecule comprising nucleotides in positions 748-933 of a mutated exon 6.

In some embodiments, an ASO as disclosed and as described hereinabove, or a pharmaceutical composition comprising thereof, is used in method for improving at least one clinical parameter of a retinal disease. In some embodiments, an ASO as disclosed and as described hereinabove, or a pharmaceutical composition comprising thereof, is used in treating of a retinal disease.

### Method of Production

According to some embodiments, there is provided a method for producing a compound suitable for treating a retinal disease.

In some embodiments, the method comprises obtaining a compound that binds to exon 6 of the NR2E3 pre-mRNA. In some embodiments, the method comprises assaying the inclusion of exon 6 of the NR2E3 pre-mRNA in the presence of the obtained compound. In some embodiments, the method comprises selecting at least one compound that induces the inclusion of exon 6 of the NR2E3 pre-mRNA. In some embodiments, inclusion is inclusion of nucleotides in positions 748-933 of the NR2E3 pre-mRNA.

In some embodiments, the method comprises obtaining a compound that binds to exon 6 of the NR2E3 pre-mRNA, and assaying the inclusion of exon 6 of the NR2E3 pre-mRNA in the presence of the obtained compound, and selecting at least one compound that induces the inclusion of exon 6 of the NR2E3 pre-mRNA, thereby producing a compound suitable for treating a retinal disease.

In some embodiments, the method comprises obtaining a compound that binds to exon 6 of the NR2E3 pre-mRNA, and assaying the inclusion of nucleotides in positions 748-933 of the NR2E3 pre-mRNA in the presence of the obtained compound, and selecting at least one compound that induces the inclusion of nucleotides in positions 748-933 of the NR2E3 pre-mRNA, thereby producing a compound suitable for treating a retinal disease.

In some embodiments, assaying is within a cell. In some embodiments, assaying is within a subject. In some embodiments, assaying is within a cell obtained or derived from a subject. In some embodiments, assaying is within a cell within a subject.

In some embodiments, assaying is in vivo. In some embodiments, assaying is in vitro or ex vivo.

In some embodiments, the method comprises obtaining a compound that binds to SEQ ID NO: 1 or SEQ ID NO: 2.

In some embodiments, the compound is an ASO. In some embodiments, the ASO is an ASO as disclosed and as described herein.

Methods of assaying exon inclusion or exclusion are common. Non-limiting examples of such methods include, but are not limited to, PCR, qPCR, gene sequencing, northern-blot, dot-blot, in situ hybridization, or others all of which would be apparent to one of ordinary skill in the art.

### General

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value (±10%). For example, a length of about 1,000 nanometers (nm) refers to a length of 1,000 nm±100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise”, or variations such as “comprises” or “comprising,” indicate the inclusion of any recited integer or group of integers but not the exclusion of any other integer or group of integers.

As used herein, the term “consists essentially of”, or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises”, “comprising”, “having” are/is interchangeable with “consisting”.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); “Monoclonal Antibodies: Methods and Protocols”. Vincent Ossipow, Nicolas Fischer. Humana Press (2014); “Monoclonal Antibodies: Methods and Protocols”. Maher Albitar. Springer Science & Business Media (2007), all of which are incorporated by reference. Other general references are provided throughout this document.

### Example 1

### Missense Mutation in Exon 6 of the NR2E3 Pre-mRNA Induces Improper Splicing Impairs Protein Nuclear Localization

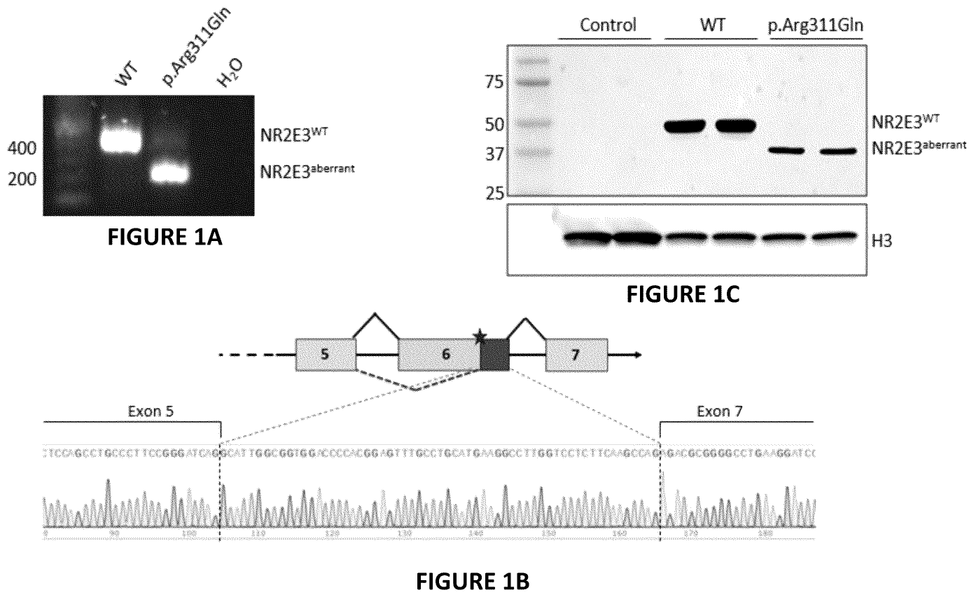

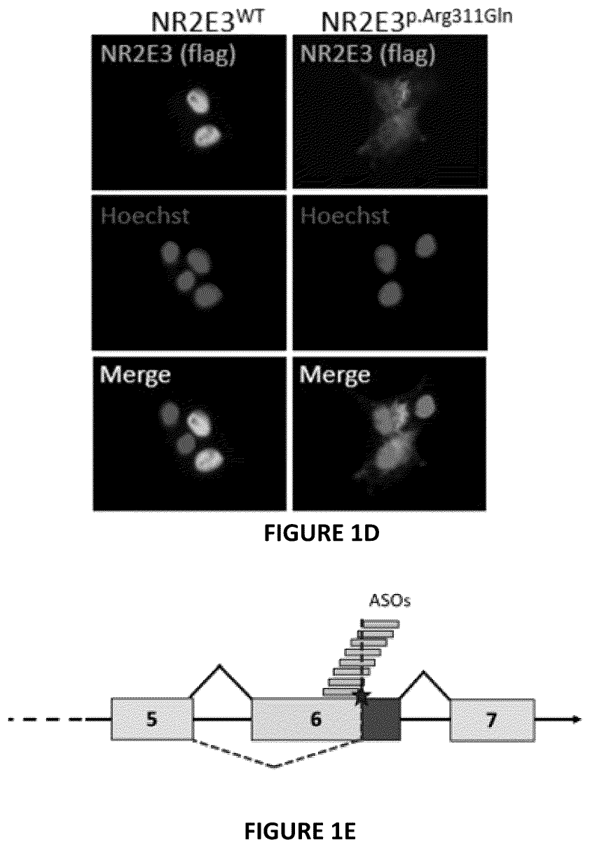

The NR2E3 gene resides on chromosome 15 (15q23), including 8 exons spanning approximately 7.7 kb of genomic DNA, and encodes a 410 amino acids protein. The c.932G>A mutation (chr15: 71813573 G>A, all genomic coordinates refer to hg38 gene model) resides in exon 6 that encodes for a part of the ligand binding domain (exon 6 genomic coordinates: chr15: 71813389-71813635). FIG. 1 demonstrates the aberrant splicing in NR2E3 gene caused by the c.932G>A mutation and a potential strategy to restore the original splicing. Using plasmids, the inventors expressed wild type (NR2E3WT) and mutant (NR2E3c.932G>A) NR2E3 in HEK293 cells. The plasmids were ordered from VectorBuilder encoding the sequence of transcript NM_014249.4, including all the exons and introns of the gene (with or without the c.932G>A mutation), and flag-tagged in the C-terminus. HEK293T cells were transfected with 1 μg of each plasmid (or control vector) using FuGene HD transfection reagent. Forty-eight (48) hours post-transfection, RNA was extracted, reverse transcribed to cDNA, and analyzed by polymerase chain reaction (PCR) using specific primers spanning exons 5 to 7. PCR results indicate that the c.932G>A mutation induced the expression of an aberrant transcript (FIG. 1A). Sequencing of the PCR product revealed that the splicing in the mutated gene is abnormal and causes the exon to start at position chr15: 71813575 (c.934G). WT exon begins at position chr15: 71813389), thus, the c.932G>A mutation leads to the loss of 186 bases (75%) of exon 6 (FIG. 1B). This result was further confirmed by western blot (WB) analysis. Protein lysates were prepared 48 hr post-transfection in RIPA buffer with protease/phosphatase inhibitor cocktail. NR2E3 expression was analyzed by WB using a specific anti-flag antibody (Sigma #F1804). The NR2E3^{c.932G>A} protein appears to be less expressed and smaller in size compared to NR2E3^{WT}, suggesting that the aberrantly spliced NR2E3^{c.932G>A} transcript is translated into a shorter, and unstable protein (FIG. 1C). The inventors further tested the sub-cellular localization of NR2E3^{c.932G>A} by immunofluorescence in HEK293 cells, following transient transfection of plasmids (48 hr), and expression of same. NR2E3^{WT} and NR2E3^{c.932G>A} proteins were detected using anti-flag antibody (Sigma-Aldrich #F1804), and the nuclei of the cells were stained with Hoechst 33342. The inventors show that whereas NR2E3^{WT} is clearly localized in the nucleus (as expected for a transcription factor), NR2E3^{c.932G>A} is characterized by a less intense staining and is mostly located outside the nucleus (FIG. 1D).

Therefore, the inventors conclude that the NR2E3^{c.932G>A} mutation leads to an aberrant splicing which may in turn relate to numerous pathological attributes, including altering the sub-cellular localization of the translated protein. Based on these results, the inventors thus suggest using ASOs to block aberrant splicing of the pre-mRNA transcribed from the mutated gene in attempt to restore normal splicing, as a potential therapeutic strategy. Such ASOs may complement and/or target any region of exon 6±50 bp up- and/or down-stream of the NR2E3 pre-mRNA transcript, including, but not limited to tens of bases up- and/or down-stream thereto, as well as the c.932G>A mutation site (FIG. 1E).

### Example 2

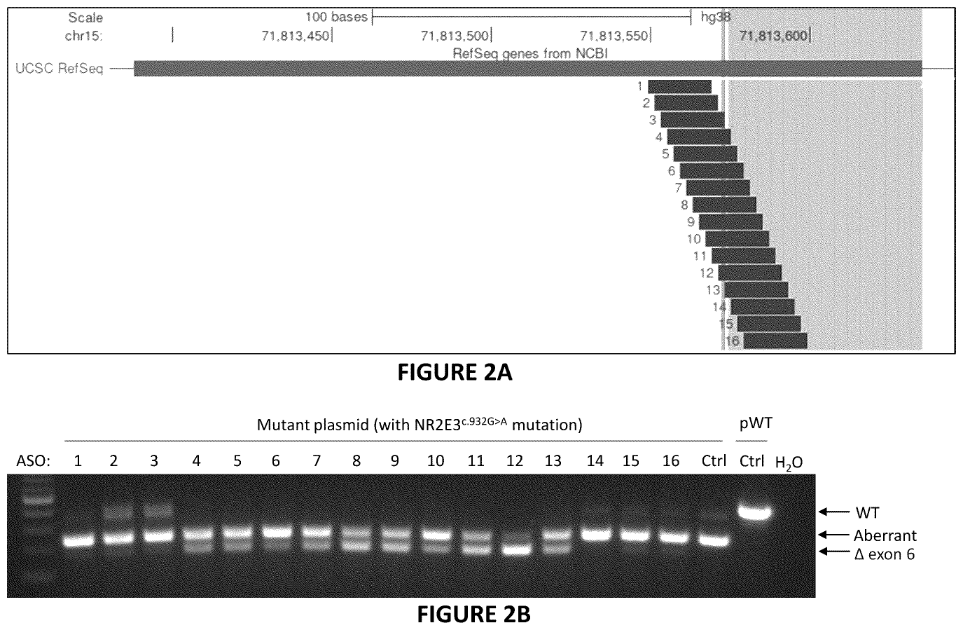

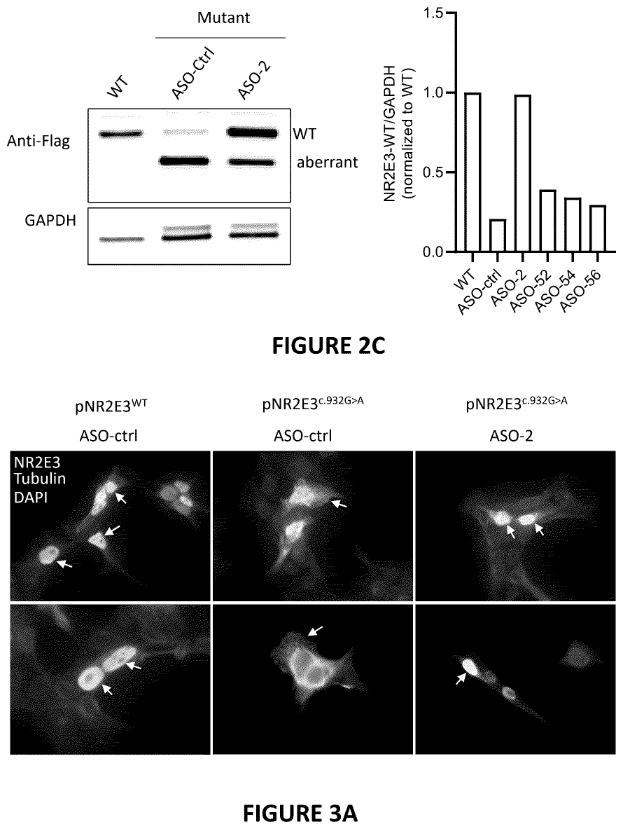

FIG. 2 depicts the design and results of an ASO screen to block aberrant splicing of NR2E3 exon 6 and restore original splicing. The inventors designed a screen of sixteen ASOs targeting the sequence of NR2E3 exon 6, specifically the region defined by SEQ ID NO: 2 (FIG. 2A). Next, the inventors performed an ASOs mini-screen using semi-quantitative PCR and identified two ASOs, ASO-2 and ASO-3 (SEQ ID NOs: 4 and 6, respectively) that significantly corrected the aberrant splicing and restored the expression of the full transcript (FIG. 2B). Further, the inventors confirmed correction of the aberrant splicing at the protein level by ASO-2 (SEQ ID NO: 4) using western blot analysis (FIG. 2C).

### Example 3

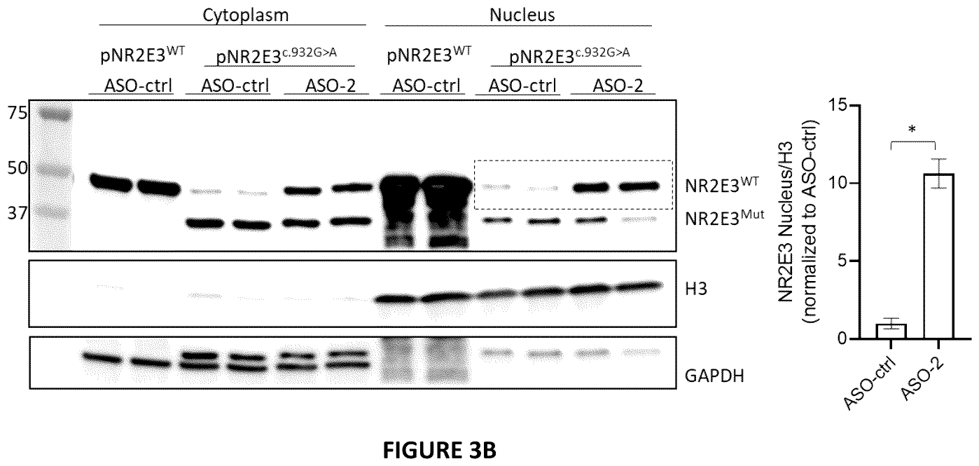

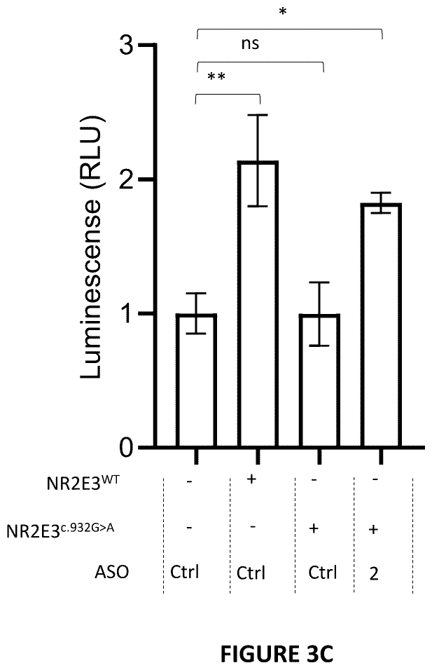

NR2E3 is known to function as a transcription factor, therefore, the inventors sought to evaluate the nuclear localization and the activation of downstream target genes of the mutant and ASO-2 treated/corrected isoforms—FIG. 3. The inventors assessed the nuclear sub-cellular localization of the NR2E3^{c.932G>A} mutant protein via immunofluorescence in HEK293 cells, following transient transfection of plasmids and found that whereas NR2E3^{WT} is clearly localized in the nucleus (as expected for a transcription factor), NR2E3.932G>A is mostly located outside the nucleus, a characteristic already reported for several other NR2E3 mutations (PMID: 19898638). Treatment with ASO-2 (SEQ ID NO: 4) restored nuclear expression of NR2E3 (FIG. 3A). The inventors further confirmed this result using nucleus-cytoplasm fractionation followed by western blot analysis. Treatment with ASO-2 (SEQ ID NO: 4) corrected aberrant splicing and restored NR2E3 protein expression in the nucleus (FIG. 3B). To demonstrate that ASO-2 (SEQ ID NO: 4) could restore the downstream activity of mutant NR2E3, the inventors assessed downstream transcription levels of Rhodopsin—a well-established downstream target gene of NR2E3 (PMID: 15689355). To this end the inventors established a luciferase reporter controlled by the rhodopsin (RHO) promoter in the presence of its co-factors CRX and NRL, using transient transfection in HEK293 cells (as described in Peng et al., 2005; Hum Mol Genet 2005 Mar. 15; 14 (6): 747-64) (FIG. 3C). Cells were additionally transfected with equal amounts of a plasmid encoding eGFP to normalize the transfection efficiency. First, the current results show that NR2E3^{c.932G>A} is unable to activate the transcription of the RHO promoter, whereas NR2E3^{WT} induces a 2-fold increase in RHO transcription activation. Treatment of NR2E3^{c.932G>A} with ASO-2 resulted in a complete rescue of RHO transcription activation (FIG. 3C), demonstrating the therapeutic potential of ASO-2 treatment.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for treating a retinal disease in a subject in need thereof, the method comprising administering to said subject a therapeutically effective amount of at least one synthetic antisense oligonucleotide (ASO), wherein said ASO induces the inclusion of nucleotides in positions 748-933 of the nuclear receptor subfamily, 2 group, E member 3 (NR2E3) pre-mRNA, thereby treating a retinal disease in the subject.

2. The method of claim 1, wherein said ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

3. The method of claim 1, wherein said ASO comprises 14 to 25 bases.

4. The method of claim 1, wherein said ASO has at least 75% complementarity to an equal-length portion of a nucleic acid sequence derived from the polynucleotide sequence:
(SEQ ID NO: 1)
GTGATCCTGCTGGAAGAGGCGTGGAGTGAACTCTTTCTCCTCGGGGCC
ATCCAGTGGTCTCTGCCTCTGGACAGCTGTCCTCTGCTGGCACCGCCC
GAGGCCTCTGCTGCCGGTGGTGCCCAGGGCCGGCTCACGCTGGCCAGC
ATGGAGACGCGTGTCCTGCAGGAAACTATCTCTCGGTTCCGGGCATTG
GCGGTGGACCCCACGGAGTTTGCCTGCATGAAGGCCTTGGTCCTCTTC
AAGCCAG.

5. The method of claim 1, wherein said ASO has at 75% to least complementarity
(SEQ ID NO: 2)
GCAGGAAACTATCTCTCGGTTCCAGGCATTGGCGGTGGACCCCACGGA
GT.

6. The method of claim 1, wherein said subject comprises at least one in-frame and/or missense mutation in exon 6 of NR2E3.

7. The method of claim 6, wherein said at least one mutation is c.932G>A.

8.-11. (canceled)

12. The method of claim 1, wherein said ASO is in a composition further comprising a pharmaceutically acceptable carrier.

13. The method of claim 1, wherein said treating comprises inducing the inclusion of nucleotides in positions 748-933 of the NR2E3 pre-mRNA, in a said subject.

14.-16. (canceled)

17. A method for producing a compound suitable for treating a retinal disease, the method comprising a compound that binds to exon 6 of the NR2E3 pre-mRNA, as saying the inclusion of nucleotides in positions 748-933 of the NR2E3 pre-mRNA in the presence of said compound, and selecting at least one compound that induces the inclusion of nucleotides in positions 748-933 of the NR2E3 pre-mRNA.

18. The method of claim 17, wherein said NR2E3 pre-mRNA comprises a c.932G>A mutation in exon 6.

19. A method for inducing inclusion of nucleotides in positions 748-933 of NR2E3 pre-mRNA in a subject in need thereof, the method comprising administering to said subject a therapeutically effective amount of at least one ASO, thereby treating a retinal disease in the subject.

20. The method of claim 19, wherein said ASO comprises a backbone selected from the group consisting of: a phosphate-ribose backbone, a phosphate-deoxyribose backbone, a phosphorothioate-deoxyribose backbone, a 2′-O-methyl-phosphorothioate backbone, a phosphorodiamidate morpholino backbone, a peptide nucleic acid backbone, a 2-methoxyethyl phosphorothioate backbone, an alternating locked nucleic acid backbone, a phosphorothioate backbone, N3′-P5′ phosphoroamidates, 2′-deoxy-2′-fluoro-β-d-arabino nucleic acid, cyclohexene nucleic acid backbone nucleic acid, tricyclo-DNA (tcDNA) nucleic acid backbone, and a combination thereof.

21. The method of claim 19, wherein said ASO comprises 14 to 25 bases.

22. The method of claim 19, wherein said ASO has at least 75% complementarity to an equal-length portion of a nucleic acid sequence derived from the polynucleotide sequence set forth in SEQ ID NO: 1.

23. The method of claim 19, wherein said ASO has at least 75% complementarity to SEQ ID NO: 2.

24. The method of claim 19, wherein said subject comprises at least one in-frame and/or missense mutation in exon 6 of NR2E3.

25. The method of claim 24, wherein said at least one mutation is c.932G>A.

26. The method of claim 19, wherein said ASO is in a composition further comprising a pharmaceutically acceptable carrier.
